(19) United States
(12) Patent Application Publication
(10) Pub. No.: US 2024/0261541 A1
(43) Pub. Date: Aug. 8, 2024

(54) MULTI-PORT DISTRIBUTION HUB FOR VASCULAR ACCESS DEVICES

(71) Applicant: Becton, Dickinson and Company, Franklin Lakes, NJ (US)

(72) Inventors: Praveen Nalawade, Belgaum (IN); Shishir Prasad, Ramsey, NJ (US)

(73) Assignee: Becton, Dickinson and Company, Franklin Lakes, NJ (US)

(21) Appl. No.: 18/163,988

(22) Filed: Feb. 3, 2023

(51) Int. Cl. A61M 25/00

(52) U.S. Cl. CPC A61M 25/0097; A61M 2005/1403; A61M 2039/0258; A61M 2039/0264; A61M 2039/0288

(57) ABSTRACT

A multi-port distribution hub for vascular access devices (VADs) facilitates sequential administration of multiple medications and VAD flushing by syringes through a catheter hub of an intravenous (IV) catheter without the need to scrub and disinfect the catheter hub after dispensing each individual syringe. A housing manifold of the hub defines a central chamber. A plurality of manifold runners each has a runner outlet in fluid communication with the central chamber, and a runner inlet in fluid communication with a corresponding syringe port. A patient access port is in fluid communication with the chamber. A one-way valve in the central chamber prevents fluid backflow through other syringe ports while dispensing fluid from one of the syringes.

### TECHNICAL FIELD

The present disclosure generally relates to multi-port distribution hubs for delivery of fluid and medication to patients via vascular access devices (VADs), including intravenous catheters.

### BACKGROUND

Vascular access devices (VAD's) are commonly used therapeutic devices and include intravenous (IV) catheters. There are two general classifications of VAD's, peripheral catheters and central venous catheters. Several types of access hubs, ports or valves are coupled to a VAD when delivering a fluid or pharmaceutical. A Luer connector is a common way to couple or join syringes, catheters, hubbed needles, IV tubes, etc. to each other. When VADs are used for medical patient care, there are potential risks of occlusions and contamination of components.

If not properly maintained, VADs can become occluded. To ensure VADs are used properly and do not become occluded, standards of practice have been developed. These standards include a cleaning procedure, which is commonly referred to as a flush procedure or flushing a catheter. VAD standards of practice usually recommend flush procedures be performed after catheter placement, before fluid infusion, and before and after drug administration, blood sampling, transfusions, and parenteral nutrition. The goal of these flush procedures is to confirm catheter patency, avoid drug incompatibilities, ensure the complete drug dose administration, prevent thrombus formation, and minimize the risk of blood stream infections. Flush procedures require diverse types and amounts of flush solutions. Commonly used flush solutions are saline and/or heparin lock solution. The type of flush solution and amount vary depending on the specific type of catheter. After flushing, the practitioner is then able to administer a dosage of medical fluid, followed by a post-administration flushing. Medication delivery and companion flushing through VAD delivery systems also inherently exposes the delivery system to potential contamination.

Bacteria and other microorganisms may gain entry into a patient's vascular system from access hubs, ports or valves upon connection to the VAD when delivering a fluid or pharmaceutical. Each access hub, port, valve or connection is associated with some risk of transmitting a catheter related bloodstream infection (CRBSI), which can be costly and potentially lethal. In order to decrease CRBSI cases and to ensure VAD's are used and maintained correctly, standards of practice have been developed, which include disinfecting and cleaning procedures. Disinfection caps have been added to the Society for Healthcare Epidemiology of America (SHEA) guidelines and caps are also incorporated into the Infusion Nurses Standards (INS) guidelines.

In developed markets, when utilizing an IV catheter, a needleless connector will typically be used to close off the system and then subsequently accessed to administer medication or other necessary fluids via the catheter to the patient. INS Standards of Practice recommend the use of a needleless connector and state that it should be “consistently and thoroughly disinfected using alcohol, tincture of iodine or chlorhexidine gluconate/alcohol combination prior to each access.” The disinfection of the needleless connector is ultimately intended to aid in the reduction of bacteria that could be living on the surface and possibly lead to a variety of catheter related complications including CRBSI. Nurses will typically utilize a 70% isopropyl alcohol (IPA) pad to complete this disinfection task by doing what is known as “scrubbing the hub.” Currently many nursing units mandate the practice of scrubbing the IV connector hub, even if the connector has a disinfection cap.

Typically, four disinfection devices and four scrubbing procedures are required to administer a single drug through a VAD system. The first disinfection scrub prepares the patient's VAD catheter hub for drug delivery and receipt of a first flushing syringe to dissolve potential occlusions within the VAD delivery system and its IV catheter. The first flushing syringe is removed after completion of the pre-flush. Thereafter a second disinfection scrub prepares the catheter hub for connection to a medication delivery syringe. A third disinfection scrub prepares the catheter hub for connection of a second flushing syringe to complete a post-medication delivery flush. Lastly, after removal of the second flushing syringe, a fourth disinfection scrub is performed, followed by sealing of the catheter hub with a cap or a locking syringe. When more than one drug is administered to a patient, each additional dose must be followed by hub disinfection before initiating the next dispensing task.

Throughout the sequence of procedures necessary to prevent occlusions within VAD systems and to administer medication to a patient there are contamination and microorganism transmission risks that can cause a CRBSI every time a syringe is connected or disconnected from the patient's VAD catheter hub. It would be desirable to minimize the number of VAD connections and disconnections needed to administer one or more drugs to a patient, as well as the number of VAD scrubbing procedures necessary to administer medication to a patient through a VAD hub or other device, in order to reduce contamination risk. Reducing the number of VAD connections and disconnections beneficially would reduce clinician time and reduce the number of syringes and disinfection caps needed to administer one or more medications to a patient.

### SUMMARY

A multi-port distribution hub for vascular access devices (VADs), as disclosed herein, facilitates sequential administration of multiple medications and VAD flushing by syringes through a catheter hub of an intravenous (IV) catheter, without the need to scrub and disinfect the catheter hub after dispensing each individual syringe. Separate syringes for each medication and for VAD flushing can be coupled to the distribution port in parallel. One-way valves in the distribution hub prevent fluid backflow through other syringe ports while dispensing fluid from one of the syringes. Various VAD delivery systems, incorporating different modular, multi-port distribution hubs, facilitate dosing of multiple medicines to a patient during a single dosing interval, or sequentially over multiple scheduled dosing intervals. By employing a distribution hub of the present disclosure, it is not required to scrub the patient's catheter hub after every separate syringe dosing. Similarly, when a distribution hub of the present disclosure is employed to flush an IV catheter and related VADs with a multi-use flushing syringe, it is not required to scrub the patient's catheter hub prior to each flushing cycle.

One aspect of the present disclosure pertains to a distribution hub for a vascular access device (VAD), which includes a housing manifold defining therein a central chamber, with a plurality of manifold runners, respectively having runner outlets on proximal ends thereof, in fluid communication with the central chamber, and respectively having runner inlets on distal ends of the manifold runners. A plurality of syringe ports is respectively in fluid communication only with a corresponding runner inlet. The housing manifold has a patient access port in fluid communication with the chamber. A one-way valve is interposed between each respective runner outlet and the central chamber, for isolating each runner from fluid backflow. In some embodiments, a unitary one-way valve is oriented within the central chamber that is biased into sealing contact all of the runner outlets and deforms or deflects or bends out of contact with any of them when fluid is dispensed out of its corresponding runner. The one-way valve biases back into contact with any outlet after fluid is dispensed out of its corresponding runner. In some embodiments, the unitary one-way valve further comprises an elastomeric tube in abutting contact with a wall of the housing that defines the central chamber, with the chamber central axis oriented coaxially with a central axis of the patient access port.

Another aspect of the present disclosure pertains to a distribution hub for a vascular access device, which includes a unistructural, homogeneous, mono-block, housing manifold that defines therein a cylindrical central chamber with a plurality of manifold runners. The manifold runners respectively have runner outlets on proximal ends thereof, in fluid communication with the central chamber, and respectively have runner inlets on distal ends thereof. A plurality of syringe ports is respectively in fluid communication only with a corresponding runner inlet. The housing manifold has a patient access port in fluid communication with the central chamber. A unitary, elastomeric tube is in abutting contact with a wall of the manifold housing that defines the central chamber. The tube is interposed between each respective runner outlet and the central chamber, for isolating each runner from fluid backflow. The tube functions as a one-way valve, which deforms or deflects or bends out of contact with any of the respective runner outlets when fluid is dispensed out of its corresponding runner. The tube biases back into contact with any outlet after fluid is dispensed out of its corresponding runner.

### BRIEF DESCRIPTION OF THE DRAWINGS

Exemplary embodiments of the disclosure are further described in the following detailed description in conjunction with the accompanying drawings, in which:

FIG. 1 is an isometric view of an embodiment of a multi-port distribution hub of the present disclosure prior to assembly within an exemplary vascular delivery system for a medical patient;

FIG. 2 is an isometric, exploded view of the distribution hub of FIG. 1;

FIG. 3 is a post-assembly, axial cross section of the distribution hub of FIG. 2, taken along 3-3 of FIG. 2;

FIG. 4 is an isometric view of the assembled vascular delivery system of FIG. 1;

FIG. 5 is a post-assembly, cross section of the distribution hub of FIG. 2, taken perpendicular to the cross section of FIG. 3, along 3-3 of FIG. 2, wherein the distribution hub is assembled within another exemplary vascular delivery system for a medical patient;

FIG. 6 is an isometric view of the post-assembly distribution hub of FIG. 2, wherein all ports of the hub are covered with peelable seals;

FIGS. 7-9 are isometric views of other embodiments of multi-port distribution hubs of the present disclosure prior to assembly within vascular delivery systems for medical patients;

FIG. 10 is an isometric view of the distribution hub of FIG. 1, assembled in a vascular delivery system that facilitates VAD flushing and medication delivery; and

FIG. 11 is a flowchart of an exemplary method for using a distribution hub of the present disclosure both to deliver medication through and flushing a VAD in a vascular delivery system for a medical patient.

To facilitate understanding, identical reference numerals have been used, where possible, to designate identical elements that are common to the figures. The figures are not drawn to scale.

### DETAILED DESCRIPTION

Use of the multi-port distribution hubs of this disclosure eliminates the need for multiple connections and disconnections to a patient's VAD involved during medication delivery. A single connection of the multi-port distribution hub to the patient's VAD facilitates parallel coupling of multiple drug delivery and flushing syringes within the patient's VAD delivery system, without risk of fluid backflow into the other hub ports. The multi-port distribution hub facilitates sequential initial flushing of the VAD delivery system for occlusion prevention, medication dosing, and post-medication delivery flushing. The multi-port hub accommodates sequential delivery of multiple drugs to the patient with only one pre-flush and post flush of the delivery system. In some embodiments, where multiple syringes remain connected to the multi-port hub, different drugs can be administered at different times in accordance with a prescribed dosing schedule while the patient port of the hub remains coupled to the patient's VAD port.

In this disclosure, where generally applicable, a convention is followed wherein the distal end of the device is the end closest to a patient, e.g., for delivery of one or more drugs to the patient through the Luer couplings disclosed herein, and the proximal end of the device is the end away from the patient and closest to a clinician or other medical practitioner. However, when describing and claiming the detailed structure of various multi-port distribution hubs disclosed herein, the terms “proximal” and “distal” also refer to orientation of inlets and outlets of manifold runners within a housing manifold of the distribution hub. Inlets on proximal ends of the manifold runners are in fluid communication with a central chamber structure of the housing manifold. Distal ends of the manifold runners are outlets that are in fluid communication with syringe ports of the housing manifold. With respect to terms used in this disclosure, the following definitions are provided.

As used herein, the use of “a,” “an,” and “the” includes the singular and plural.

As used herein, the term “Luer connector” refers to a connection collar that is the standard way of attaching syringes, catheters, hubbed needles, IV tubes, etc. to each other. The Luer connector consists of male and female interlocking tubes, slightly tapered to hold together better with even just a simple pressure/twist fit. Some Luer connectors embodiments include an additional outer rim of threading, allowing them to be more securely coupled to mating threaded connectors. The Luer connector male end is associated with a flush syringe and can interlock and connect to the female end located on the vascular access device (VAD). A Luer connector also has a distal end channel that releasably attaches the Luer connector to the hub of a VAD, and a proximal end channel that releasably attaches the Luer connector to the barrel of a syringe.

As used herein, ISO 80369-7:2016 defines a specification for standard Luer connectors including a 6% taper between the distal end and the proximal end. A male standard Luer connector increases from the open distal end to the proximal end. A female standard Luer connector decreases from the open proximal end to the distal end. According to ISO 80369-7:2016, a male standard Luer connector has an outer cross-sectional diameter measured 0.75 mm from the distal end of the tip of between 3.970 mm and 4.072 mm. The length of the male standard Luer taper is between 7.500 mm to 10.500 mm. The outer cross-sectional diameter measured 7.500 mm from the distal end of the tip is between 4.376 mm and 4.476 mm. As used herein, the phrases “male standard Luer connector” and “female standard Luer connector” shall refer to connectors having the dimensions described in ISO 80369-7, which is hereby incorporated by reference in its entirety.

As would be readily appreciated by skilled artisans in the relevant art, while descriptive terms such as “tip”, “hub”, “thread”, “protrusion/insert”, “tab”, “slope”, “wall”, “top”, “side”, “bottom” and others are used throughout this specification, as applicable, to facilitate understanding, it is not intended to limit any components that can be used in combinations or individually or to require specific spatial orientations, to implement various aspects of the embodiments of the present disclosure.

Before describing several exemplary embodiments of the disclosure, it is to be understood that the disclosure is not limited to the details of construction or process steps set forth in the following description. The disclosure is capable of other embodiments and of being practiced or being conducted in many ways. For example, where the Luer couplings disclosed herein have first and second mating fitting portions shown in one configuration (e.g., mating male and female connections), in other embodiments, the location and orientation of the mating fitting portions are reversed.

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

In an exemplary implementation of the embodiments of present disclosure, a first fitting of the Luer connector includes a distal end having a needleless connection Luer connector. In one or more embodiments, the needleless connection includes at least one thread and other features in all combinations allowing it to interface with a corresponding thread or plurality of threads of a corresponding connector.

According to still further exemplary implementations of the embodiments of the present disclosure, the collar or the needleless connector may bend or elastically deform to allow better interference fit compliance with corresponding connectors.

According to still further exemplary implementations of the embodiments of the present disclosure, the needleless connector may comprise female threads that are sized and have a thread pattern that will engage with a standard ISO594-2 type of male fitting and/or male threads that are sized and have a thread pattern that will engage with a standard ISO594-2 type of female fitting. An example of an ISO594-2 type of fitting is a Q-style fitting.

In one or more embodiments, a male or female connector may be selected from the group consisting essentially of: needle-free connectors, catheter Luer connectors, stopcocks, and hemodialysis connectors. In one or more embodiments, the needleless connector is selected from a Q-Syte connector, MaxPlus, MaxPlus Clear, MaxZero, UltraSite, Caresite, InVision-Plus, Safeline, OneLink, V-Link, ClearLink, NeutraClear, Clave, MicroClave, MicroClave Clear, Neutron, NanoClave, Kendall, Nexus, InVision, Vadsite, Bionector, etc. In one or more embodiments, the male connector or coupling may be an intravenous tubing end, or a stopcock coupled to a port of the Luer coupling.

Before describing several exemplary embodiments of the disclosure, it is to be understood that the disclosure is not limited to the details of construction or process steps set forth in the following description. The disclosure is capable of other embodiments and of being practiced or being carried out in various ways.

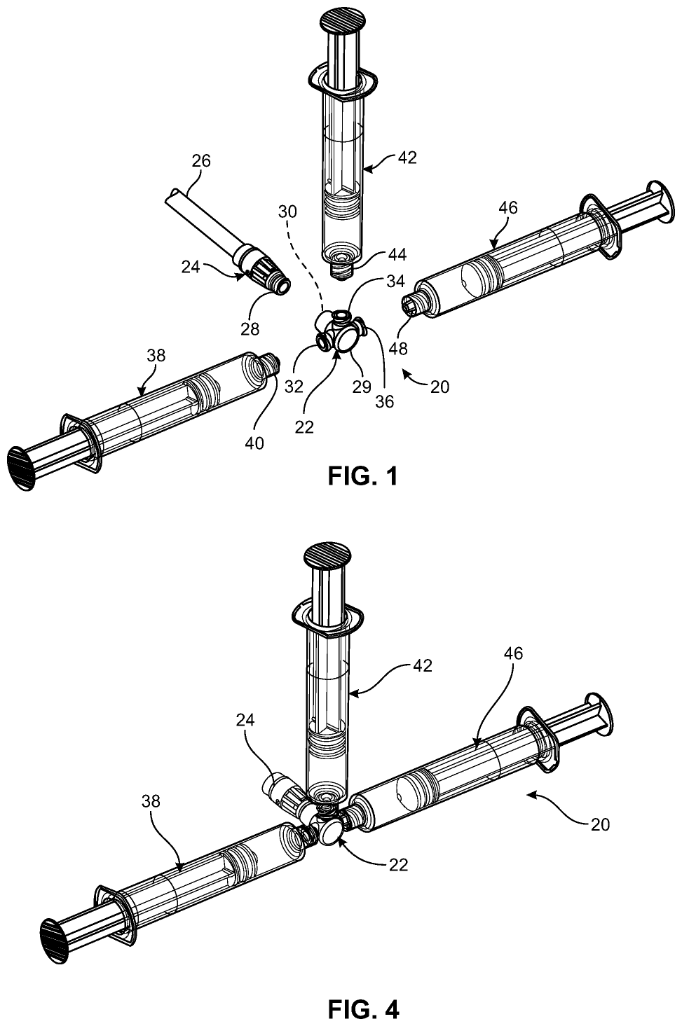

Referring generally to FIGS. 1-4, a first embodiment of a vascular access device (VAD) delivery system 20 includes a distribution hub 22 for coupling to a catheter port-type VAD hub 24. The catheter port VAD 24 is in turn coupled to and in fluid communication with an IV catheter line 26 for insertion into a patient. As shown in FIGS. 1 and 4, a threaded male Luer connector 28 of the catheter port VAD 24 is coupled to a patient access port 30 of the distribution hub 22.

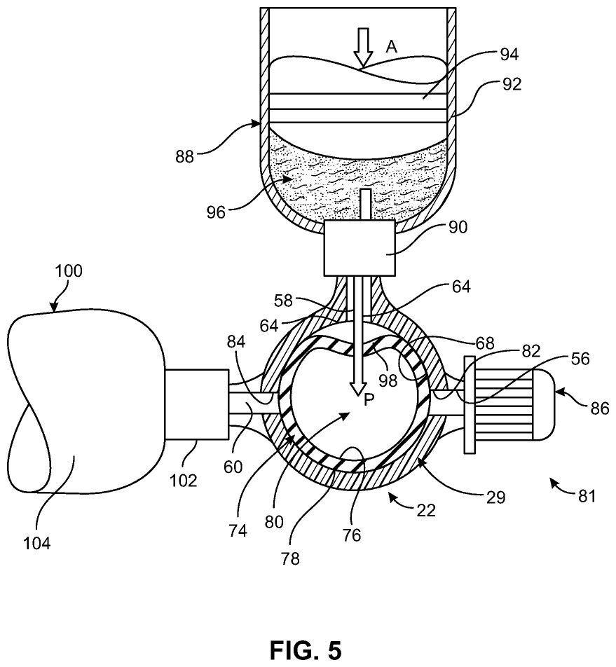

The distribution hub 22 comprises a unistructural, homogeneous, mono-block, housing manifold 29, fabricated by molding medical-grade plastic polymer. The housing manifold 29 is also utilized in the VAD delivery system embodiment of FIG. 5. In other embodiments, the housing manifold is fabricated by joining subcomponent portions. The housing manifold 29 defines the aforementioned patient access port 30 as well as respective first 32, second 34 and third 36 syringe ports. In other embodiments, the housing manifold defines two syringe ports. In yet other embodiments, the housing manifold defines more than three syringe ports. A first syringe 38 has a threaded Luer connector 40 that is coupled to a first corresponding manifold-Luer connector 50 of the first syringe port 32, defined by the housing manifold 29. A second syringe 42 has a threaded Luer connector 44 that is coupled to a second corresponding manifold-Luer connector 52 of the second syringe port 34. A third syringe 46 has a threaded Luer connector 48 that is coupled to a corresponding third manifold-Luer connector 54 of the third syringe port 36, defined by the housing manifold 29. Thus, as shown in FIG. 4, the distribution hub 22 is capable of simultaneous, parallel coupling of three syringes 38, 42 and 44 to a VAD 24 for a patient.

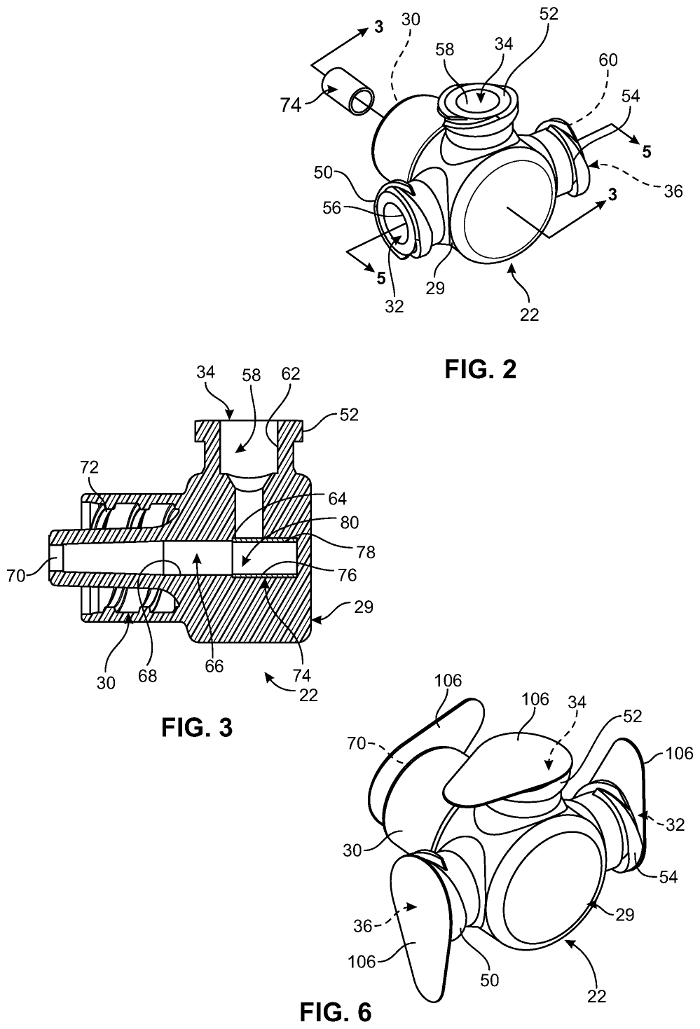

FIGS. 2, 3 and 5 depict internal fluid communication pathways within the housing manifold 29, which are applicable in any VAD delivery system employing that housing manifold. A first manifold runner 56 is in fluid communication with the first syringe port 32. Correspondingly, a second manifold runner 58 is in fluid communication with the second syringe port 34 and a third manifold runner 60 is in fluid communication with the third syringe port 36. FIG. 3 illustrates the complete internal fluid communication pathway of the second manifold runner 58, wherein its inlet 62 is formed at the juncture of the opening of the second syringe port 34 and the corresponding second manifold-Luer connector 52. An outlet 64 of the second manifold runner 58 is in fluid communication with a central chamber 66. Chamber wall surface 68 defines the central chamber 66 volume. Each of the other first 56 and third 60 manifold runners also has an inlet in fluid communication with its corresponding first 32 and third 36 syringe port and an outlet in fluid communication with the central chamber 66. In this description, a proximal end of each respective manifold runner 56, 58, 60 is its outlet into the adjoining the central chamber 66 and a distal end of each respective manifold runner is its inlet from its adjoining, corresponding manifold-Luer connector 50, 52, 54.

In the embodiment of distribution hub 22 and its housing manifold 29, the profile of the central chamber 66 is cylindrical and defines a chamber central axis that is coaxial with a central axis of the patient access port 30. The central chamber 66 is in fluid communication with a patient port outlet 70 of the patient access port 30. The patient access port 30 incorporates a threaded Luer connector 72 for coupling to a corresponding threaded Luer connector of a VAD, such as the Luer connector 28 of the VAD 24. In other embodiments, the central chamber does not have a cylindrical profile and is not oriented coaxially with the central axis of the patient access port. In other embodiments, the patient access port has a connector that is not a threaded Luer connector.

In the various distribution hub embodiments of this disclosure, including the distribution hub 22, a one-way valve is interposed between each respective runner outlet and the central chamber, for isolating each runner from fluid backflow. In the distribution hub 22 and its housing manifold 29 shown in FIGS. 2, 3 and 5, the one-way valve 74 is a unitary, elastomeric tube, oriented within the central chamber 66, with an inner diameter surface 76 facing the chamber and an outer diameter surface 78 that is in abutting contact with the central chamber wall surface 68. The inner diameter surface 76 of the tubular one-way valve 74 defines a lumen 80, which along with the chamber wall surface 68 co-defines the cavity of the central chamber 66. The one-way valve 74 is constructed with medical-grade elastomer.

Exemplary function of the one-way valve 74 is shown in FIG. 5 with respect to a second embodiment of a VAD delivery system 81 that utilizes the same housing manifold 29 of the first VAD delivery system embodiment 20 of FIGS. 1 and 4. The outer diameter wall surface 78 of the of one-way valve 74 abuts and isolates each of the second outlet 64 of the second manifold runner 58, the first outlet 82 of the first manifold runner 56 and the third outlet 84 of the third manifold runner 60 from communication with the central chamber 66. The tubular, elastomeric, one-way valve 74 deforms or deflects or bends in a radially inwardly direction toward the central axis of the central chamber 66, out of contact with any of the respective runner outlets 64, 82, 84 when pressurized fluid P is dispensed out of its corresponding runner but isolates any other non-pressurized manifold runners to prevent backflow into them. The one-way valve 74 biases back into contact with any runner outlet 64, 82, 84 after fluid is dispensed out of its corresponding runner.

In the VAD delivery system 81 of FIG. 5, the first manifold runner 56 of the housing manifold 29 is capped with a Luer cap 86, with the one-way valve 74 isolating the first manifold outlet 82 from communication with the one-way valve lumen 80, the central chamber 66 and the patient port outlet 70. A threaded Luer connector 90 of syringe 88 is coupled to the second manifold runner 58. Syringe barrel 92 retains a syringe stopper 94 that is advancing (arrow A) and dispensing medication 96, as depicted by the pressurized fluid arrow P. A portion 98 of tubular one-way valve 74 deforms or deflects or bends radially inwardly, out of contact with the corresponding second runner outlet 64, in response to pressure force P generated by the syringe-dispensed medication fluid 96. The one-way valve 74 deflection at the portion 98 allows the medication 96 to be dispensed into the lumen 80 of the one-way valve. After the medication 96 is dispensed and the pressure drops in the manifold runner 58, the one-way valve 74 biases back into contact with the runner outlet 64. Referring also to FIG. 3, medication 96 thereafter flows into the central chamber 66 and exits the outlet 70 of the patient access port 30 for dispensing to a patient. Syringe 100 is coupled to the third manifold runner 60 by Luer connector 102 that is formed at the distal end of its syringe barrel 104. The one-way valve 74 isolates the third manifold outlet 84 from communication with the one-way valve lumen 80, the central chamber 66 and the patient port outlet 70.

In some embodiments, one or more of the ports of the housing manifold is sealed with a peel tab or a hub cap prior to sterile packaging, to inhibit contamination of distribution hub. In FIG. 6, the outlet 70 as well as the entire internal surfaces of the patient access port 30 is sealed with an adhesive peel tab 106. The respective first 32, second 34 and third 36 syringe ports are also covered with respective adhesive peel tab 106 affixed to the respective first 50, second 52 and third 54 manifold Luer connectors. In one or more embodiments, the peelable seal 106 comprises an aluminum or multi-layer polymer film peel back top. In a specific embodiment, the peelable seal 106 is heat-sealed or induction sealed to the end face of the cap to seal the open proximal end. In one or more embodiments, the peelable seal 106 comprises a moisture barrier. In FIG. 5, a Luer cap 86 sealed the corresponding first manifold runner 56 and its corresponding syringe port.

Distribution hub embodiments of this disclosure incorporate plural syringe ports, varying in number and in spatial orientation about their housing manifolds. Advantageously, a clinician utilizes a distribution hub with a sufficient number of syringe ports to administer all required medications for a prescribed schedule time. By doing so, a clinician may couple plural syringes in any dosing sequence about the distribution hub, so that the medication in each is dosed in conformity with the sequence merely by dispensing each syringe serially along the hub in a consistent direction (e.g., left to right; clockwise or counterclockwise). Also advantageously, coupling all syringes to the hub prior to coupling to the patient's catheter port reduces likelihood of error of missing or doubling a medication dose. Alternately, the hubs are pre-marked sequentially by the manufacturer for easy remembrance by the clinician of the steps completed during, e.g., the medication delivery process. For example, if clinician attention to the patient is diverted after delivering using the medication syringe, the clinician can look at the number of hubs used to remind themself of flushing the line before the subsequent procedure. Also, distribution hub of the present disclosure may also be used in the home care setting. Since the workflow becomes very simple to implement compared to current SASH workflow, it will provide added advantage for homecare setting.

In some embodiments, such as the distribution hub 22 of FIGS. 1-6, the respective distal ends of the first 56, second 58, and third 60 manifold runners radially project away from, and are aligned along a plane that is perpendicular to the central axis of the central chamber 66. In other embodiments various manifold runners are not in planar alignment.

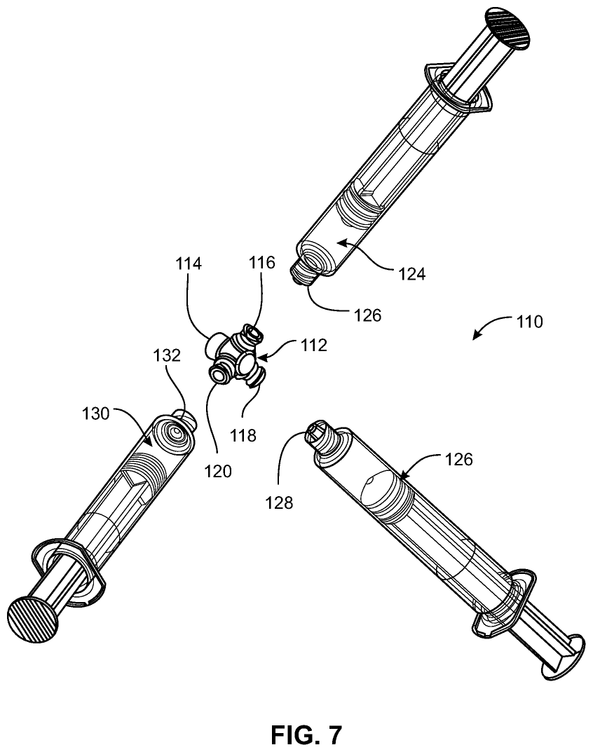

In the VAD delivery system 110 embodiment of FIG. 7, the distribution hub 112 comprises a patient access port 114 that is oriented coaxially with the central axis if its central chamber. The distal ends of respective manifold runners that are coupled to each of its corresponding respective first 116, second 118 and third 120 syringe ports radially project away from the chamber and the patient access port 114 in non-planar orientation. In this embodiment, a first syringe 122 will be coupled to the first syringe port 116 by syringe Luer connector 124, a second syringe 126 will be connected to the second syringe port 118 by syringe Luer connector 128 and a third syringe 130 will be connected to the third syringe port 120 by syringe Luer connector 132.

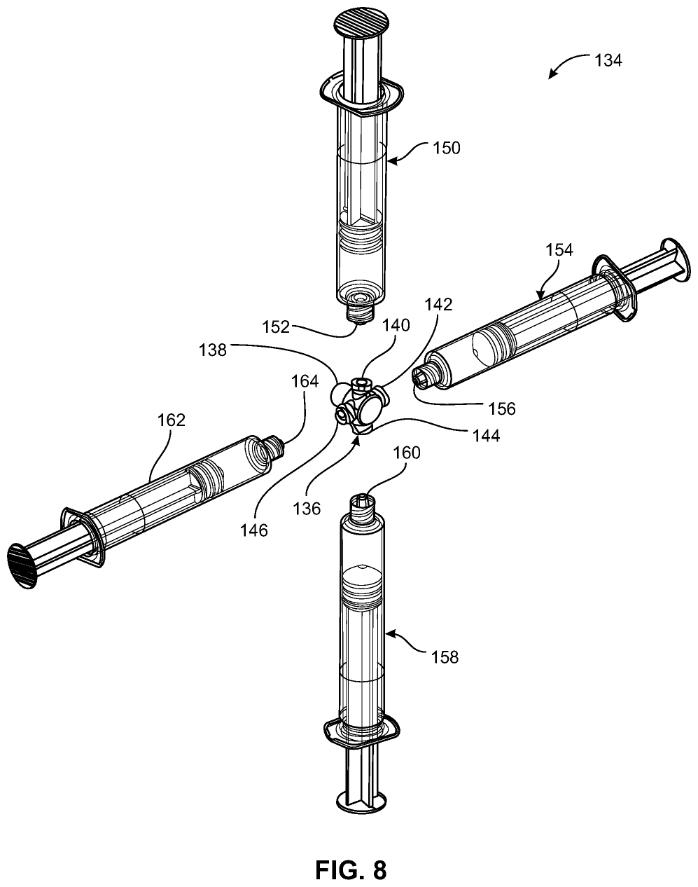

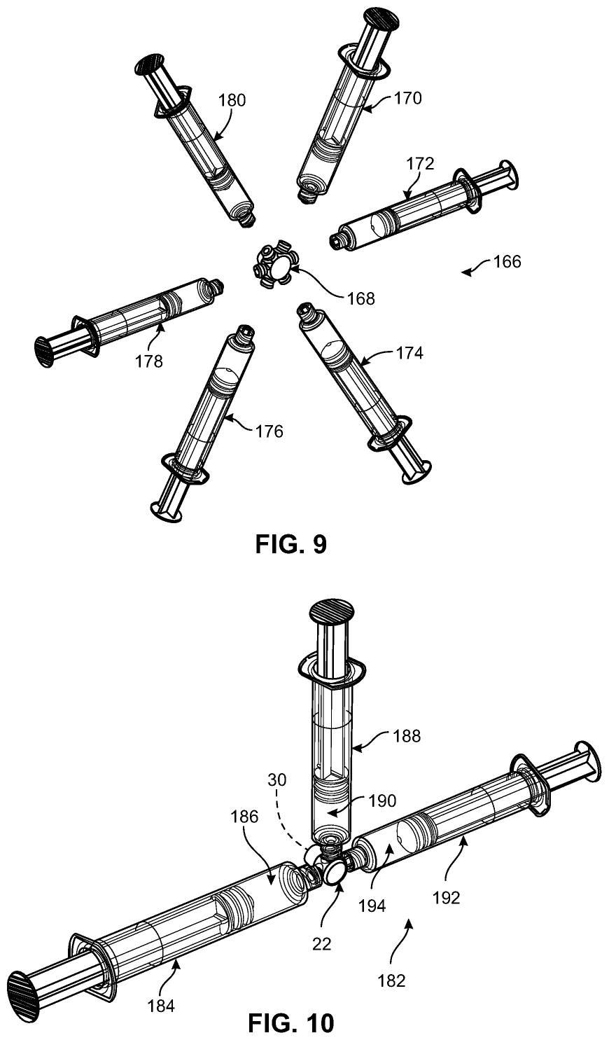

In the VAD delivery system 134 embodiment of FIG. 8, the distribution hub 136 comprises a patient access port 138 that is oriented coaxially with the central axis if its central chamber. The distal ends of respective manifold runners that are coupled to each of its corresponding respective first 140, second 142, third 144 and fourth 146 syringe ports radially project away from the chamber and the patient access port 138 in a planar orientation. In this embodiment, a first syringe 150 will be coupled to the first syringe port 140 by syringe Luer connector 152, a second syringe 154 will be connected to the second syringe port 142 by syringe Luer connector 156, a third syringe 158 will be connected to the third syringe port 144 by syringe Luer connector 160, and a fourth syringe 162 will be connected to the fourth syringe port 146 by syringe Luer connector 164. In the VAD delivery system 166 embodiment of FIG. 9, the distribution hub 168 has six syringe ports for coupling of up to 6 syringes 170, 172, 174, 176, 178, and 180.

In all of the syringe distribution hub embodiments 22, 112, 144 and 168 shown in FIGS. 1-9 of this disclosure, each comprises a housing manifold defining therein a central chamber; a plurality of manifold runners, respectively having runner outlets on proximal ends thereof, in fluid communication with the central chamber, and respectively having runner inlets on distal ends thereof; a plurality of syringe ports respectively in fluid communication only with a corresponding runner inlet; and a patient access port in fluid communication with the chamber. Each of those hub embodiments also comprises a one-way valve corresponding to and interposed between each respective runner outlet and the central chamber, for isolating each runner from fluid backflow. The distribution hub embodiment 22 generally depicts fluid flow paths and the one-way valve interposed between each manifold runner and central chamber that are present in all of the other embodiments 112, 144 and 168. The differences between the various embodiments are primarily the number and relative orientation of syringe ports about the housing manifold of each respective distribution hub. While the hub embodiment 22 incorporates a tubular construction one-way valve 74, in other embodiments, separate one-way valves are interposed between each manifold runner outlet and the central chamber.

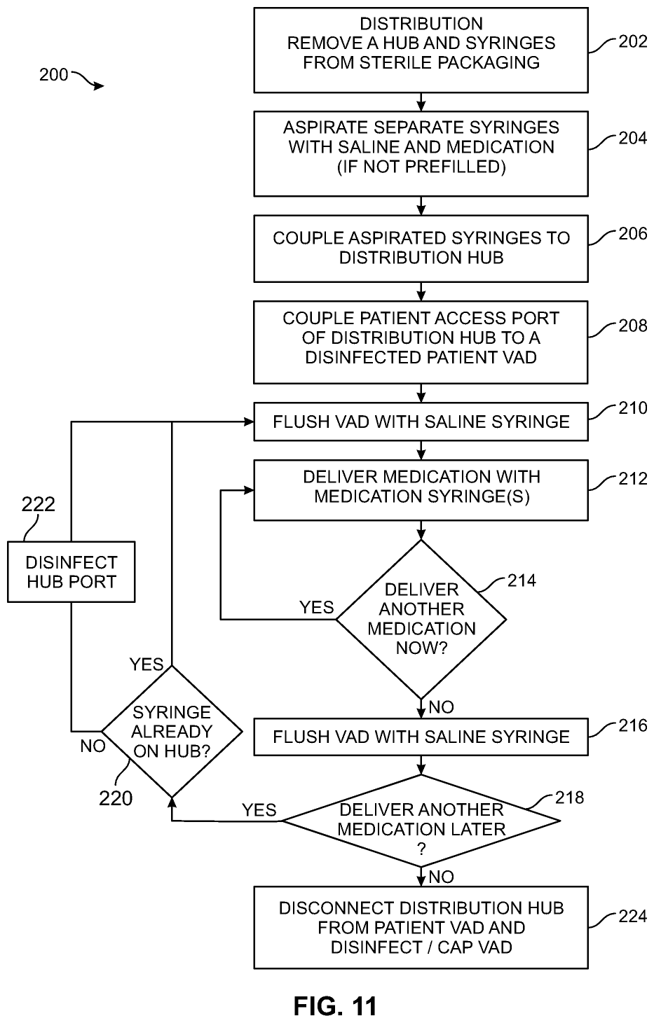

An exemplary way and method for using the distribution hub embodiment 22 in a VAD delivery system 182, for flushing a VAD and subsequent delivery of medications, while minimizing the number hub sterilization steps and number of administration syringes, is shown in FIGS. 10 and 11. Referring to FIG. 10, after the VAD delivery system 182 is fully assembled, the patient access port 30 of the distribution hub 22 is intended to be coupled to a venous catheter or other VAD (not shown). A flush syringe 184, containing flushing solution such as saline, is coupled to one of the syringe ports of the distribution hub 22. The flush syringe 184 contains sufficient flushing solution to perform at least two complete flushes of the distribution hub 22 and all downstream VAD components to the patient, in conformity with clinical practices. In some embodiments, the flush syringe 184 contains sufficient flushing solution to perform more than two VAD flushes. A first medication syringe 188, containing a first medication 190, and a second medication syringe 192, containing a second medication 194 are also coupled to other separate, respective syringe ports of the distribution hub 22.

In some embodiments, one or more of the syringes 184, 188 and 192 are prefilled with their intended medicinal fluids prior to sterile syringe packaging, in order to reduce on-site dispensing and sterilization tasks at the treatment site. Alternatively, in some embodiments, the syringes are coupled to the distribution hub 22 after coupling the patient access port 30 to the patient's VAD. In other embodiments, some syringes are coupled to the distribution hub 22 prior to coupling the patient access port 30 to the patient's VAD, and thereafter other syringes are coupled to the distribution hub.

Referring to the flowchart 200 of FIG. 11, at step 202, a clinician removes a distribution hub 22, the syringes 184, 190 and 192 and any other required VADs from their respective sterile packaging, in conformity with clinical practices. Next, at step 204, unless the sterilized syringes 184, 190 and 192 are prefilled, a clinician aspirates them with their required flushing solution or medication, applying any needed disinfection protocols to medication vials or syringe fill needles prior to contact with the syringes. The sterilized Luer connectors of the aspirated syringes 184, 190 and 192 are coupled to their respective syringe ports of the sterilized distribution hub 22 at step 206. At step 208, after manual disinfection of the patient VAD with sterilization wipes and disinfectant fluid or a scrub cap, it is coupled to the access port 30 of the sterilized distribution hub 22.

After coupling the patient access port 30 to the patient's VAD (e.g., a catheter hub), the clinician flushes the patient's VAD with the saline syringe 184, at step 210. Medication is delivered to the patient from one or both syringes 190, 192 at step 212. Referring to the decision step 214, if for example, medication is only to be delivered from syringe 190 under a prescribed dosage time, then the clinician proceeds to flush the patient's VAD with the saline syringe 184 a second time at step 216. Conversely, the second VAD flushing is not performed if medication in the second syringe 192 is also to be administered to the patient at the same prescribed dosage time as that of syringe 190. The second VAD flush is performed at step 214 until after the last medication dose is administered at the prescribed dosage time.

Referring to the decision step 218, if other syringes are already coupled to the distribution hub 22 for delivery at a later time, the patient access port 30 remains coupled to the patient's VAD until the scheduled time. When one or more of the remaining medication syringes that is already on the hub is to be delivered (see decision step 220), the clinician repeats the VAD flush of step 210, the medication delivery 212 and the second VAD flush of step 216. Advantageously, to reduce risk of CRBSI incidences, the distribution hub 22 is treated as a single use device and discarded after delivery and flushing of all medication syringes originally attached to it. Alternatively, at decision step 218, if a clinician chooses to re-use an existing port on the distribution hub to couple to it an additional, new medication syringe, rather than discard the existing distribution hub 22, the re-used port is disinfected at step 222, followed by the VAD flush of step 210, the medication delivery 212 and the second VAD flush of step 216. Referring to step 224, after all medication syringes coupled to the distribution hub 22 (or any distribution hub with more than three syringe ports) have been dispensed and a decision has been made to remove the distribution hub from the patient's VAD, the patient access port 30 of the distribution hub and its attached, used syringes 184, 190 and 192 are disconnected from the patient's VAD, and discarded in conformance with biohazard disposal protocols. Then, patient's VAD is disinfected and capped.

In following the VAD flushing and medication administration protocols with the pre-sterilized distribution hub 22 and the pre-sterilized syringes 184, 190 and 192 as single use devices, only two manual disinfection scrub cycles are required; namely, scrub the patient VAD prior to its coupling to and subsequent to decoupling from the distribution hub 22. Recall that under prior existing protocols, administration of a single medication injection through a patient's VAD required at least four manual disinfection scrub cycles, namely: (1) initial scrubbing of the VAD, (2) post initial flush scrubbing, (3) post medication scrubbing after each serial drug delivery (i.e., once before each new syringe injection), and (4) post second flush scrubbing upon completion of all medication dispensing. Benefits of the distribution hubs of the present disclosure to minimize disinfection scrubbing cycles are more readily apparent when more than two drugs are administered to a patient in a dispensing cycle with multiple, parallel syringes sharing a common distribution hub, because the patient VAD does not need to be scrubbed after each separate syringe dispenses its medication.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein provided a description with reference to particular embodiments, it is to be understood that these embodiments are merely illustrative of the principles and applications of the disclosure. It will be apparent to those skilled in the art that various modifications and variations can be made to the present disclosure without departing from the spirit and scope thereof. Thus, it is intended that the present disclosure include modifications and variations that are within the scope of the appended claims and their equivalents.

What is claimed is:

1. A multi-port distribution hub for a vascular access device (VAD), comprising:
a housing manifold defining therein:
a central chamber;
a plurality of manifold runners, respectively having runner outlets on proximal ends thereof, in fluid communication with the central chamber, and respectively having runner inlets on distal ends thereof;
a plurality of syringe ports respectively in fluid communication only with a corresponding runner inlet; and
a patient access port in fluid communication with the chamber; and
a one-way valve corresponding to and interposed between each respective runner outlet and the central chamber, for isolating each runner from fluid backflow.

2. The distribution hub of claim 1, further comprising housing defining a cylindrical central chamber having a chamber central axis.

3. The distribution hub of claim 2, further comprising a unitary one-way valve, oriented within the central chamber that is biased into sealing contact all of the runner outlets and deflects out of contact with any of them when fluid is dispensed out of its corresponding runner.

4. The distribution hub of claim 3, the unitary one-way valve further comprising an elastomeric tube in abutting contact with a wall of the housing that defines the central chamber, with the chamber central axis oriented coaxially with a central axis of the patient access port.

5. The distribution hub of claim 2, further comprising the plurality of manifold runners aligned along a plane that is perpendicular to the housing central axis.

6. The distribution hub of claim 2, further comprising the respective distal ends of the plurality of manifold runners radially projecting away from the chamber.

7. The distribution hub of claim 1, further comprising the respective distal ends of the plurality of manifold runners radially projecting away from the chamber.

8. The distribution hub of claim 1, further comprising at least three manifold runners and respective corresponding syringe ports.

9. The distribution hub of claim 1, the patient access port comprising a catheter port.

10. The distribution hub of claim 1, the patient access port comprising a threaded Luer connector.

11. The distribution hub of claim 1, the plurality of syringe ports comprising Luer connectors.

12. The distribution hub of claim 1, the plurality of syringe ports comprising threaded Luer connectors.

13. The distribution hub of claim 1, further comprising at least one peel tab covering at least one of the syringe ports and/or the patient access port.

14. The distribution hub of claim 1, at least one of the respective one-way valves comprising a pliable membrane oriented within the central chamber that is biased into sealing contact with a runner outlet and deflects out of contact therewith when fluid is dispensed out of its corresponding runner.

15. The distribution hub of claim 1, further comprising a unitary one-way valve that is biased into sealing contact all of the runner outlets and deflects out of contact with any of them when fluid is dispensed out of its corresponding runner.

16. The distribution hub of claim 15, the unitary one-way valve comprising an elastomeric tube.

17. The distribution hub of claim 16., the housing defining a cylindrical central chamber having a chamber central axis that is coaxial with a central axis of the patient access port.

18. A multi-port distribution hub for a vascular access device, comprising:
a unistructural, homogeneous, mono-block, housing manifold defining therein:
a cylindrical central chamber;
a plurality of manifold runners, respectively having runner outlets on proximal ends thereof, in fluid communication with the central chamber, and respectively having runner inlets on distal ends thereof;
a plurality of syringe ports respectively in fluid communication only with a corresponding runner inlet; and
a patient access port in fluid communication with the central chamber; and
a unitary, elastomeric tube in abutting contact with a wall of the housing that defines the central chamber, the tube interposed between each respective runner outlet and the central chamber, for isolating each runner from fluid backflow, the tube functioning as a one-way valve, which deflects out of contact with any of the respective runner outlets when fluid is dispensed out of its corresponding runner.

19. The distribution hub of claim 18, the syringe ports and/or the patient access port further comprising a threaded Luer connector.

20. The distribution hub of claim 19, further comprising at least three manifold runners, each with a corresponding threaded Luer connector-type, syringe access port.

21. A method for flushing a vascular access device (VAD) and administering plural medications to a patient through the VAD, through the multi-port distribution hub of claim 20, comprising:
inserting an IV catheter, having a VAD catheter hub, into a vein of a patient;
providing a sterile, multi-port distribution hub having plural syringe ports;
coupling a sterile flush syringe containing flushing fluid to a first one of the syringe ports, and a sterile medication delivery syringe containing medication to a second one of the syringe ports;
coupling the patient access port of the distribution hub to a disinfected VAD hub of the IV catheter;
flushing the VAD with the flush syringe;
administering the medication with the medication deliver syringe;
re-flushing the VAD with the same flush syringe without removing same from its first syringe port;
decoupling the patient access port of the distribution hub from the mating VAD catheter hub; and
disinfecting and capping the VAD catheter hub.
